(19) United States
(12) Patent Application Publication
(10) Pub. No.: US 2019/0029531 A1
(43) Pub. Date: Jan. 31, 2019

(54) METHOD, APPARATUS, AND SYSTEM FOR DIAGNOSING PERICARDIAL TAMPONADE

(71) Applicant: RUSH UNIVERSITY MEDICAL CENTER, Chicago, IL (US)

(72) Inventor: Rami DOUKKY, Oak Park, IL (US)

(73) Assignee: RUSH UNIVERSITY MEDICAL CENTER, Chicago, IL (US)

(21) Appl. No.: 16/050,729

(22) Filed: Jul. 31, 2018

Related U.S. Application Data
(60) Provisional application No. 62/539,335, filed on Jul. 31, 2017.

(51) Int. Cl. A61B 5/0205; A61B 5/1455; A61B 5/021; A61B 5/00

(52) U.S. Cl. CPC A61B 5/0205; A61B 5/1455; A61B 5/02108; A61B 5/6816; A61B 5/6826; A61B 5/742; A61B 5/7235

(57) ABSTRACT

The disclosure is directed to a method, apparatus, and system for diagnosing pericardial tamponade. Pulse oximetry is a noninvasive method for monitoring a person's oxygen saturation. The method includes analyzing a pulse oximetry waveform of a person and obtaining an oximetry paradoxus corresponding to the variation of the pulse oximetry waveform, which includes identifying a maximum amplitude and a minimum amplitude of the pulse oximetry waveform within a respiratory cycle and calculating a ratio of the maximum amplitude and the minimum amplitude to obtain the oximetry paradoxus. The method also includes determining whether the oximetry paradoxus is equal to or larger than a threshold. When the oximetry paradoxus is determined to be equal to or larger than the threshold, the method identifies the person having pericardial tamponade. Therefore, the disclosure provides a noninvasive, inexpensive, and ubiquitous tool in aiding in the accurate diagnosis of pericardial tamponade.

### RELATED APPLICATION

This application claims priority to Provisional Application No. 62/539,335, filed on Jul. 31, 2017, which is incorporated by reference in its entirety.

### BACKGROUND

### 1. Technical Field

A method, apparatus, and system for diagnosing pericardial tamponade are provided and in particular, the method, apparatus, and system use pulse oximetry waveform variation analysis to obtain an oximetry paradoxus.

2. Background Information

Pericardial tamponade is a life-threatening condition, caused by pathologic fluid accumulation in the pericardial space, increasing intra-pericardial pressure and compression of the cardiac chambers leading to a hemodynamic collapse. Having a high index of suspicion and rapid diagnosis are essential for timely evacuation of the pericardial fluid to avoid or treat cardiovascular collapse and imminent death. Pericardial tamponade, when suspected, can be confirmed by echocardiography or right heart catheterization. However, these diagnostic tools are often not readily available at the point-of-care. The symptoms and signs of tamponade include dyspnea, chest discomfort, tachycardia, tachypnea, hypotension, elevated jugular venous pulsation, and pulsus paradoxus. Unfortunately, most of these are nonspecific and can be encountered in a multitude of cardiopulmonary conditions. Pulsus paradoxus is defined as excessive respiratory variation in the systolic blood pressure (≥10 mmHg). Pulsus paradoxus is considered pathognomonic for clinically significant pericardial tamponade, but is also seen in asthma and chronic obstructive pulmonary disease (COPD) exacerbation. Nonetheless, pulsus paradoxus is often difficult to demonstrate by inexperienced clinicians or less skilled healthcare personnel. Therefore, there is a need for a simple, reliable, rapid, reproducible, and ubiquitous bedside diagnostic tool that can aid clinicians in identifying patients with evolving pericardial tamponade.

Pulse oximetry is a noninvasive method for monitoring a person's oxygen saturation. Pulse oximetry plethysmographic waveform demonstrates amplitude variations under certain conditions. As described below in the present disclosure, by analyzing the variation of the amplitude of the pulse oximetry waveform, a new method for diagnosing pericardial tamponade may be performed, providing a noninvasive, inexpensive, and ubiquitous tool in aiding in the accurate diagnosis of pericardial tamponade.

### BRIEF SUMMARY

The present disclosure describes a method for diagnosing pericardial tamponade. The method includes analyzing, by a device comprising a memory and a processor in communication with the memory, a pulse oximetry waveform of a person to obtain an oximetry paradoxus. For the step of analyzing the pulse oximetry waveform to obtain the oximetry paradoxus, the method includes identifying, by the device, a maximum amplitude and a minimum amplitude of the pulse oximetry waveform within a respiratory cycle, and calculating, by the device, a ratio of the maximum amplitude and the minimum amplitude to obtain the oximetry paradoxus. The method also includes determining, by the device, whether the oximetry paradoxus is equal to or larger than a threshold. When the oximetry paradoxus is determined to be equal to or larger than the threshold, the method further includes identifying, by the device, the person as having or suspected as having pericardial tamponade.

The present disclosure also describes a device for diagnosing pericardial tamponade. The device includes a memory storing instructions, and a processor in communication with the memory, wherein, when the processor executes the instructions, the processor is configured to cause the device to identify a maximum amplitude and a minimum amplitude of a pulse oximetry waveform of a person within a respiratory cycle. The processor is also configured to cause the device to calculate a ratio of the maximum amplitude and the minimum amplitude to obtain an oximetry paradoxus. The processor is also configured to cause the device to determine whether the oximetry paradoxus is equal to or larger than a threshold, and when the oximetry paradoxus is determined to be equal to or larger than the threshold, identify the person as having or suspected as having pericardial tamponade.

The present disclosure further describes a non-transitory computer readable storage medium. The non-transitory computer readable storage medium includes instructions for diagnosing pericardial tamponade. When the instructions are executed by a processor in a device, the instructions direct the device to identify a maximum amplitude and a minimum amplitude of a pulse oximetry waveform of a person within a respiratory cycle and calculate a ratio of the maximum amplitude and the minimum amplitude to obtain an oximetry paradoxus. When the instructions are executed by a processor in a device, the instructions also direct the device to determine whether the oximetry paradoxus is equal to or larger than a threshold, and when the oximetry paradoxus is determined to be equal to or larger than the threshold, identify the person as having or suspected as having pericardial tamponade.

The present disclosure also describes a method for diagnosing pericardial tamponade. The method includes analyzing a pulse oximetry waveform of a person to obtain an oximetry paradoxus, and determining whether the oximetry paradoxus is equal to or larger than a threshold. When the oximetry paradoxus is determined to be equal to or larger than the threshold, the method further includes identifying the person as having or suspected as having pericardial tamponade.

### BRIEF DESCRIPTION OF THE DRAWINGS

FIG. 1 is a pulse oximetry plethysmographic waveform within one respiratory cycle.

FIG. 2A is a pulse oximetry waveform of a person.

FIG. 2B shows a pulse oximetry waveform of a person before undergoing pericardial window.

FIG. 2C shows a pulse oximetry waveform of the person after undergoing pericardial window.

FIG. 2D shows a pulse oximetry waveform of another person.

FIG. 3 shows an example of a flow diagram of a method for diagnosing pericardial tamponade.

FIG. 4 shows an example a flow diagram of steps that may be included in the method for diagnosing pericardial tamponade.

FIG. 5 shows an example of a commercially available pulse oximetry and cardiac monitor device.

FIG. 6 shows subjects with pericardial tamponade having a statistically higher mean oximetry paradoxus.

FIG. 7A shows a receiver operator characteristic (ROC) curve for all patients.

FIG. 7B shows a receiver operator characteristic (ROC) curve for an oximetry paradoxus and a pulsus paradoxus.

FIG. 7C shows a receiver operator characteristic (ROC) curve for a derivation cohort.

FIG. 7D shows a receiver operator characteristic (ROC) curve for a validation cohort.

FIG. 8 shows an oximetry paradoxus pre and post drainage of pericardial fluid.

### DETAILED DESCRIPTION OF THE DRAWINGS

The invention will now be described in detail hereinafter with reference to the accompanied drawings, which form a part of the present invention, and which show, by way of illustration, specific examples of embodiments. Please note that the invention may, however, be embodied in a variety of different forms and, therefore, the covered or claimed subject matter is intended to be construed as not being limited to any of the embodiments to be set forth below. Please also note that the invention may be embodied as methods, devices, components, or systems. Accordingly, embodiments of the invention may, for example, take the form of hardware, software, firmware or any combination thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter includes combinations of exemplary embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

Pulse oximetry is routinely performed in emergency rooms, hospitals and clinics. The pulse oximetry instrument may be a commercially available instrument, which includes but is not limited to a Passport 2-Datascope pulse oximetry and cardiac monitor device (Datascope Corp; Fairfield N.J.). The pulse oximetry instrument can measure the oximetry signal and display or output a pulse oximetry plethysmographic waveform, which is also known as a pulse oximetry waveform or simply as a pulse oximetry. The pulse oximetry sensor may be placed on the patient's body, for example, the patient's index finger or earlobe.

The oximetry signal depends on the capillary volume, which is dependent on stroke volume and arterial pressure. Therefore, an amplitude of the pulse oximetry waveform may vary with the respiratory cycle in conditions such as pericardial tamponade, thus demonstrating an objective graphic representation of pulsus paradoxus. Thus, analyzing the amplitude of the pulse oximetry waveform may provide a simple, reliable, rapid, reproducible, and ubiquitous bedside diagnostic tool that can aid clinicians in identifying patients with pericardial tamponade.

A variation index can be quantified by analyzing the variation of the pulse oximetry waveform of a person within respiratory cycles, and this variation index may be compared with a threshold to determine whether the person has pericardial tamponade. The variation index may be quantified based on quantification of elements of the pulse oximetry waveform.

In one embodiment, the variation index may be calculated as a difference of the amplitudes between the maximum amplitude and minimum amplitude within a respiratory cycle. When the pulse oximetry waveform comprises multiple respiratory cycles, the variation index may also be calculated as an average or weighted average of the differences of the amplitudes for all or a subset of multiple respiratory cycles.

In another embodiment, the variation index may be calculated as a ratio of the amplitudes between the maximum amplitude and minimum amplitude within a respiratory cycle. When the pulse oximetry waveform comprises multiple respiratory cycles, the variation index may also be calculated as an average or weighted average of the ratios of the amplitudes for all or a subset of multiple respiratory cycles.

When the pulse oximetry waveform comprises multiple respiratory cycles, the averaged ratio calculated from the above calculation method may be different from a method of calculating a ratio of the amplitudes between the maximum amplitude and minimum amplitude not limited to one respiratory cycle.

For example, the pulse oximetry waveform comprises two respiratory cycles. Two method will be used to calculate a ratio and their corresponding result will be compared.

1. For a first method of calculating a ratio of the amplitudes between the maximum amplitude and minimum amplitude within a respiratory cycle, i.e., the maximum amplitude and minimum amplitude are within the same respiratory cycle. During the first respiratory cycle, for example, a maximum amplitude is 0.8 and a minimum amplitude is 0.5, and thus, the ratio for the first respiratory cycle is calculated as 0.8/0.5=1.6. During the second respiratory cycle, for example, a maximum amplitude is 0.6 and a minimum amplitude is 0.4, and thus, the ratio for the second respiratory cycle is calculated as 0.6/0.4=1.5. Therefore, a simple averaged ratio for the pulse oximetry waveform including the two respiratory cycles is calculated to be (1.6+1.5)/2=1.55.

2. For a second method of calculating a ratio of the amplitudes between the maximum amplitude and minimum amplitude not limited to one respiratory cycle. For the above exemplary pulse oximetry waveform including two respiratory cycles, a maximum amplitude is 0.8 and a minimum amplitude is 0.4 for the pulse oximetry waveform including two respiratory cycles. Thus, a ratio for the pulse oximetry waveform is calculated to be 0.8/0.4=2.

As shown above, the ratio calculated from the above two methods have very different results (1.55 vs. 2), which may lead to different analysis and determinations.

In another embodiment, the variation index may be calculated as a difference of areas between the maximum area and minimum area below the pulse oximetry waveform within a respiratory cycle. When the pulse oximetry waveform comprises multiple respiratory cycles, the variation index may also be calculated as an average or weighted average of the difference of the areas for all or a subset of multiple respiratory cycles.

In another embodiment, the variation index may be calculated as a ratio of areas between the maximum area and minimum area below the pulse oximetry waveform within a respiratory cycle. When the pulse oximetry waveform comprises multiple respiratory cycles, the variation index may also be calculated as an average or weighted average of the ratios of the areas for all or a subset of multiple respiratory cycles.

The variation index of the pulse oximetry waveform may be standardized in diagnosing pericardial tamponade. The standardized variation index may be validated in a separate cohort. The measurement and calculation of the variation index may be implemented with software applications, so that the variation index may be obtained automatically, giving automated alert of possibility of pericardial tamponade. In the right clinical context, this can be followed by confirmatory diagnostic testing, allowing for timely lifesaving intervention to evacuate the fluid around the heart causing the condition.

This disclosure is a major improvement over existing tools of diagnosing pericardial tamponade. Pulsus Paradoxus is one existing clinical test to measure respiratory variation in systolic blood pressure by measuring a change of blood pressure during respiration. However, performing the pulsus paradoxus test requires a high level of clinical skill. Most non-cardiologists cannot perform this test properly. Almost all nursing and support staff cannot properly conduct this test either. Even if they could, it takes certain level of suspicion of pericardial tamponade to conduct the test since pulsus paradoxus measurement is not part of routine care.

Echocardiograph is another existing tool for diagnosing pericardial tamponade. Echocardiography is somewhat expensive, not readily available at the point of care and requires specialized technologist to perform the test and a trained cardiologist to interpret the test result. Echocardiography is often unavailable off hours and on weekends. Echocardiography takes approximately an hour (under best circumstances) to order, perform, and interpret an echocardiogram. The physician should suspect pericardial tamponade in order to request an echocardiogram.

The methods and systems described herein require minimal to no training to conduct and minimal skill to interpret. The test takes minutes to perform. The test of the present disclosure can raise suspicion of the condition at which time a confirmatory test can be performed, for example, an echocardiogram, and a definitive treatment can be delivered. With the implemented software application in the present disclosure, the test of present disclosure can be completely automated to provide alerts of possible cardiac tamponades within seconds. The present disclosure can be performed routinely on all patients, and its use does not incur any additional cost or efforts.

FIG. 1 shows a pulse oximetry plethysmographic waveform 100 within one respiratory cycle, including an x-axis 190, which is typically time, and a y-axis 192, which typically corresponds to a pulse oximetry signal intensity. The pulse oximetry waveform may include at least two cardiac cycles, for example, two, three, five cardiac cycles. The pulse oximetry waveform 100 in FIG. 1 contains four complete cardiac cycles, including a first cardiac cycle 140 and a second cardiac cycle 180.

In one embodiment, an amplitude of the pulse oximetry waveform with a cardiac cycle can be calculated. For example, in the first cardiac cycle 140, the pulse oximetry waveform has a peak 110, which is the highest oximetry signal point within the first cycle 140, and a nadir 120, which is the lowest oximetry signal point following the peak 110 within the first cycle 140. The amplitude 130 of the first cycle 140 is calculated as the difference of oximetry signal between the peak 110 and the nadir 120.

An amplitude of the pulse oximetry waveform for any other cardiac cycle can be calculated in a similar way. For example, the pulse oximetry waveform of the second cardiac cycle 180 has a peak 150, which is the highest oximetry signal point within the second cycle 180, and a nadir 160, which is the lowest oximetry signal point following the peak 150 within the second cycle 180. The amplitude 170 of the second cycle 180 is calculated as the difference of oximetry signal between the peak 150 and the nadir 160.

Among the four complete cardiac cycles in FIG. 1, the amplitude 130 of the first cycle 140 is the smallest (i.e., minimum) amplitude, and the amplitude 170 of the second cycle 180 is the largest (i.e., maximum) amplitude.

In one embodiment, the variation index can be calculated as a ratio of the largest (or larger if the pulse oximetry waveform only contains two cardiac cycles) amplitude of the pulse oximetry waveform and the smallest (or smaller if the pulse oximetry waveform only contains two cardiac cycles) amplitude of the pulse oximetry waveform within the respiratory cycle. The ratio (i.e., the variation index) may be also called the oximetry paradoxus ratio, or simply the oximetry paradoxus. In FIG. 1, the oximetry paradoxus may be obtained by calculating the ratio of the maximum amplitude 170 of the second cardiac cycle 180 and the minimum amplitude 130 of the first cardiac cycle 140.

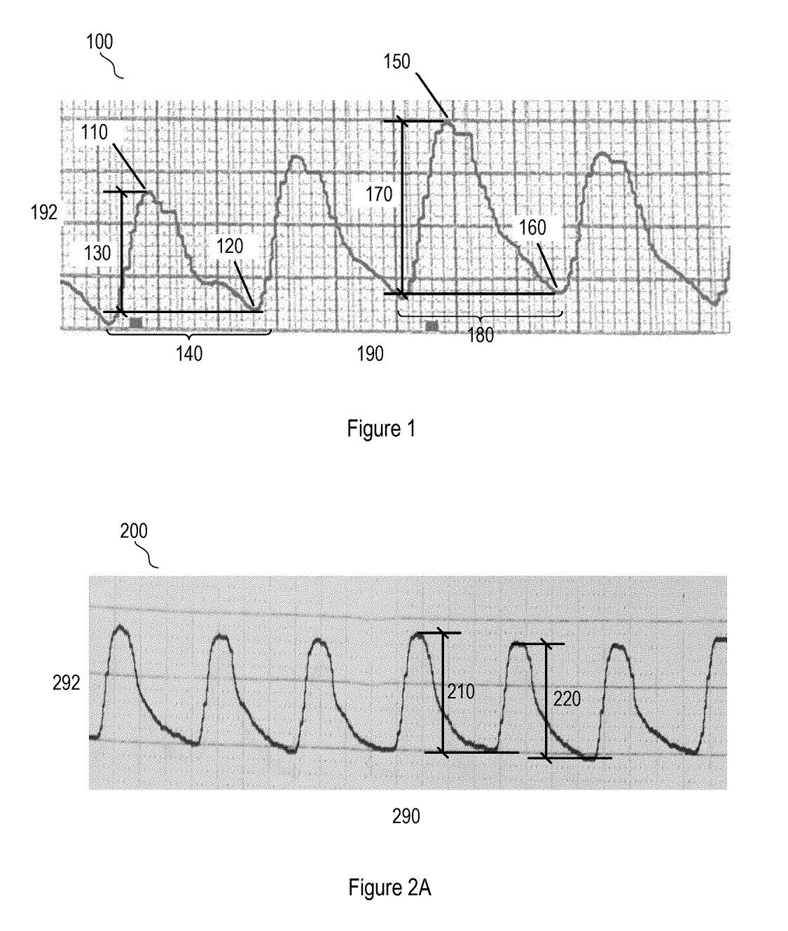

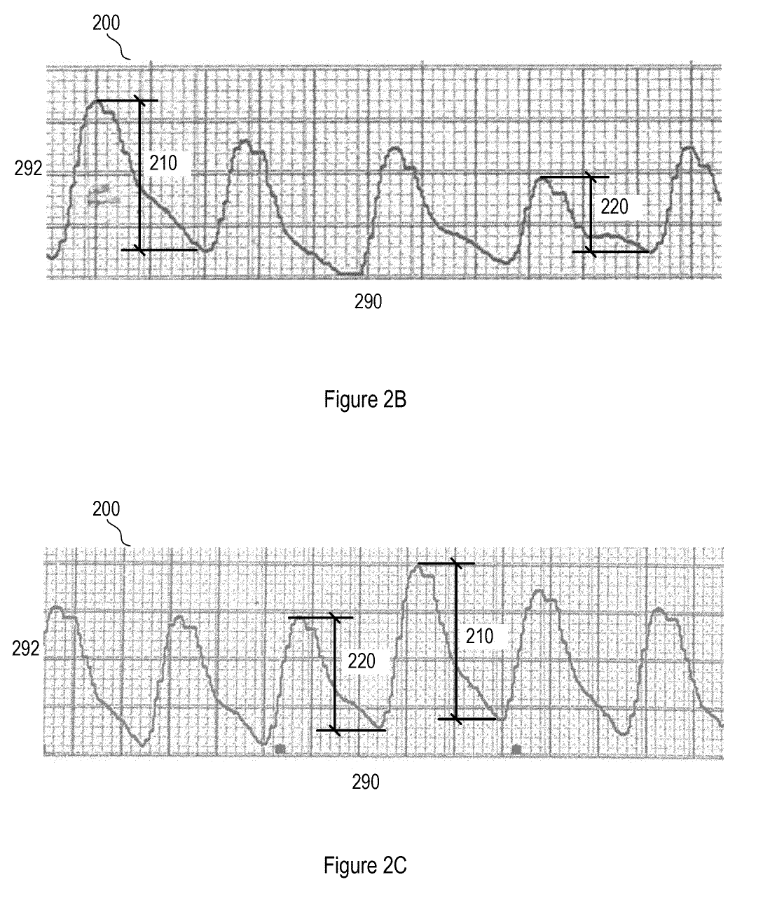

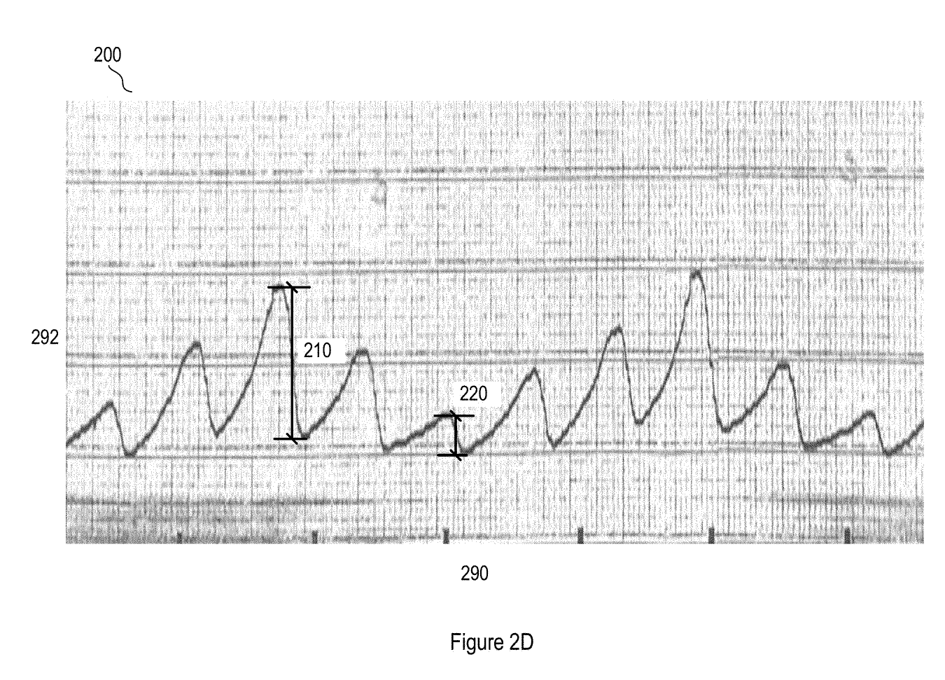

FIG. 2A shows a pulse oximetry waveform 200 of a person, including an x-axis 290, which is typically time, and a y-axis 292, which typically corresponds to a pulse oximetry signal intensity. The pulse oximetry waveform 200 in FIG. 2A includes six complete cardiac cycles. The amplitude 210 is the maximum amplitude among all cardiac cycles in FIG. 2A, and the amplitude 220 is the minimum amplitude among all cardiac cycles in FIG. 2A. The oximetry paradoxus for FIG. 2A may be obtained by calculating the ratio of the maximum amplitude 210 and the minimum amplitude 220.

FIG. 2B shows a pulse oximetry waveform 200 of a person before undergoing a pericardial window procedure, including an x-axis 290, which is typically time, and a y-axis 292, which typically corresponds to an oximetry signal intensity. The pulse oximetry waveform 200 in FIG. 2B includes four complete cardiac cycles. The amplitude 210 is the maximum amplitude among all cardiac cycles in FIG. 2B, and the amplitude 220 is the minimum amplitude among all cardiac cycles in FIG. 2B. The oximetry paradoxus for FIG. 2B may be obtained by calculating the ratio of the maximum amplitude 210 and the minimum amplitude 220.

FIG. 2C shows a pulse oximetry waveform 200 of the person after undergoing the pericardial window procedure, including an x-axis 290, which is typically time, and a y-axis 292, which typically corresponds to an oximetry signal intensity. The pulse oximetry waveform 200 in FIG. 2C includes four complete cardiac cycles. The amplitude 210 is the maximum amplitude among all cardiac cycles in FIG. 2C, and the amplitude 220 is the minimum amplitude among all cardiac cycles in FIG. 2C. The oximetry paradoxus for FIG. 2C may be obtained by calculating the ratio of the maximum amplitude 210 and the minimum amplitude 220.

FIG. 2D shows a pulse oximetry waveform 200 of another person, including an x-axis 290, which is typically time, and a y-axis 292, which typically corresponds to an oximetry signal intensity. The pulse oximetry waveform 200 in FIG. 2D includes ten complete cardiac cycles. The amplitude 210 is the maximum amplitude among all cardiac cycles in FIG. 2D, and the amplitude 220 is the minimum amplitude among all cardiac cycles in FIG. 2D. The oximetry paradoxus for FIG. 2D may be obtained by calculating the ratio of the maximum amplitude 210 and the minimum amplitude 220.

In other embodiments, the pulse oximetry waveform may contain more than one respiratory cycle. Then, a ratio of the maximum and minimum amplitudes may be calculated for each respiratory cycle, and the oximetry paradoxus for the pulse oximetry waveform is an average of all ratios from the multiple respiratory cycles.

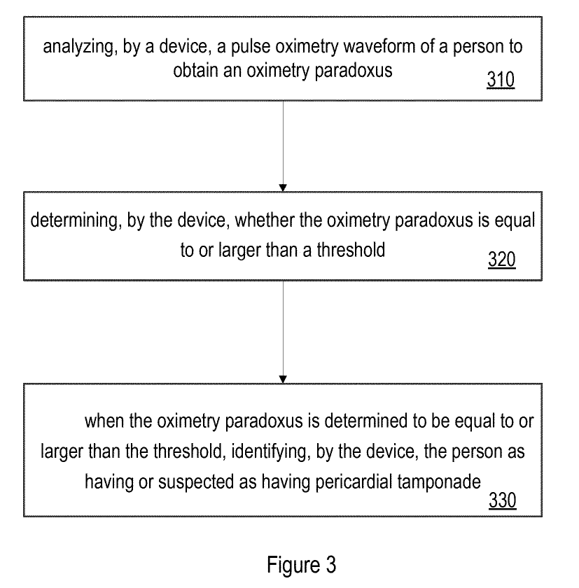

FIG. 3 shows a method for diagnosing pericardial tamponade. The method includes step 310: analyzing, by a device, a pulse oximetry waveform of a person to obtain an oximetry paradoxus; step 320: determining, by the device, whether the oximetry paradoxus is equal to or larger than a threshold; and step 330: when the oximetry paradoxus is determined to be equal to or larger than the threshold, identifying, by the device, the person as having or suspected as having pericardial tamponade.

Before the step 310 analyzing a pulse oximetry waveform of a person, the pulse oximetry waveform of the person may be obtained by a pulse oximeter. The pulse oximetry waveform may contain one or more respiratory cycles, and each respiratory cycles may contain one or more cardiac cycles. In another embodiment, the pulse oximetry waveform may be analyzed while the pulse oximetry waveform is being obtained by the pulse oximeter to shorten the time needed to perform the disclosed method.

In step 310, a device is configured to analyze a pulse oximetry waveform of a person and obtain the oximetry paradoxus corresponding to the pulse oximetry waveform. The device may include a memory for storing instructions and a processor in communication with the memory. When the processor executes the instructions, the processor is configured to cause the device to perform specific acts, such as but not limited to analyzing a pulse oximetry waveform. The person may be a healthy person or a patient. Based on the results of the analysis, the person may be suspected as having pericardial tamponade, or may be identified as having pericardial tamponade. In some embodiments, a second diagnostic test may be performed in persons suspected of having or identified as having pericardial tamponade.

In one embodiment, the pulse oximetry waveform may be part of one respiratory cycle and contain two cardiac cycles, including a first cardiac cycle and a second cardiac cycle. The device is configured to analyze the pulse oximetry waveform to obtain a first amplitude of the first cardiac cycle and a second amplitude of the second cardiac cycle, respectively. The device is also configured to identify a larger amplitude and a smaller amplitude between the first amplitude and the second amplitude. Then the device is configured to calculate the ratio of the larger amplitude and the smaller amplitude to obtain the oximetry paradoxus.

In another embodiment, the pulse oximetry waveform may be part of one respiratory cycle and contain more than two cardiac cycles, for example, three, five, or any other number of cardiac cycles. The device is configured to analyze the pulse oximetry waveform and obtain an amplitude for each cardiac cycle. The device is also configured to identify the largest (i.e., maximum) amplitude and the smallest (i.e., minimum) amplitude among all amplitudes for all cardiac cycles. Then the device is configured to calculate the ratio of the maximum amplitude and the minimum amplitude to obtain the oximetry paradoxus.

During obtaining a pulse oximetry waveform, a pulse oximeter is very susceptible to errors, resulting in errors in the pulse oximetry waveform, for example, a pulse oximeter probe or sensor moves due to the motion of the person. To mitigate this issue, the pulse oximetry waveform may contain at least two respiratory cycles, for example, two, three, five, or any other number of respiratory cycles. The pulse oximetry waveform within each respiratory cycle may contain at least two cardiac cycles. For each respiratory cycle of the pulse oximetry waveform, the device is configured to analyze each respiratory cycle and identify the largest (or larger if only two cardiac cycles in this particular respiratory cycle) amplitude and the smallest (or smaller if only two cardiac cycles in this particular respiratory cycle) amplitude within each respiratory cycle. Then the device is configured to calculate a ratio of the largest (or larger) amplitude and the smallest (or smaller) amplitude for each respiratory cycle of the pulse oximetry waveform. Furthermore, the device may be configured to calculate an average of all ratios of all respiratory cycles to determine the oximetry paradoxus.

In step 320, the device is configured to determine whether the oximetry paradoxus is equal to or larger than a threshold. The oximetry paradoxus may be a number between about 1 and about 3.3, and the threshold may be any number between about 1 and about 3.3. The larger the threshold is, the lower the sensitivity of diagnosing pericardial tamponade is. Additionally, the larger the threshold is, the higher the specificity of diagnosing the pericardial tamponade is.

In one embodiment, the threshold may be a “sensitive” threshold with ≥90% sensitivity, or a “specific” threshold with ≥80% specificity. In another embodiment, the threshold may be about 1.24 with sensitivity at or approaching 100%, about 1.48 with highest accuracy, or about 1.74 with specificity >90%.

In step 330, the device is further configured to, when the oximetry paradoxus is determined to be equal to or larger than the threshold, identify the person having pericardial tamponade. In another embodiment, when the oximetry paradoxus is determined to be equal to or larger than the threshold, the method includes identifying, by the device, the person as having or suspected as having pericardial tamponade with a sensitivity and a specificity. In one embodiment, the threshold may be about 1.24, the corresponding sensitivity is about 100%, and the corresponding specificity is about 61%. The threshold may be about 1.48, the corresponding sensitivity is about 78%, and the corresponding specificity is about 93%. The threshold may also be about 1.74, the corresponding sensitivity is about 33%, and the corresponding specificity is about 93%.

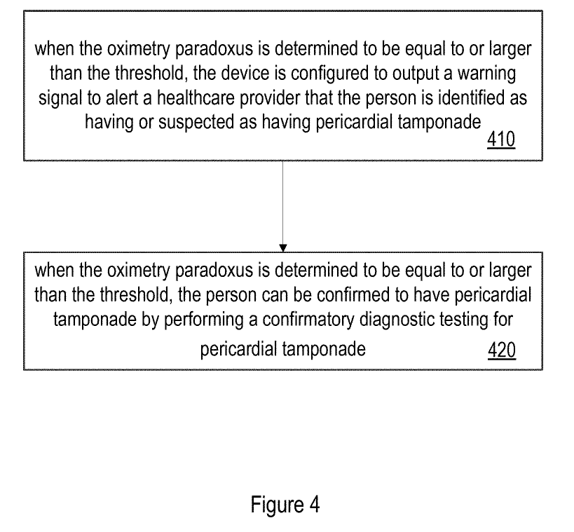

FIG. 4 shows steps that may be included in the method for diagnosing pericardial tamponade, including step 410: when the oximetry paradoxus is determined to be equal to or larger than the threshold, the device is configured to output a warning signal to alert a healthcare provider that the person is identified as having or suspected as having pericardial tamponade.; In some embodiments, step 420 may be included wherein when the oximetry paradoxus is determined to be equal to or larger than the threshold, confirmation of pericardial tamponade may be made by performing a confirmatory diagnostic testing for pericardial tamponade.

In step 410, when the oximetry paradoxus is determined to be equal to or larger than the threshold, the device is configured to output the warning signal to alert a healthcare provider that the person is identified to have pericardial tamponade. The corresponding sensitivity and specificity may or may not be included in the warning signal. The warning signal may include an audio signal, for example, a beeping sound. The warning signal may include a light signal, for example, a flashing red light to alert the healthcare provider. Alternatively, the warning signal may include a video signal, for example, a flashing text or image on the display of the device to alert the healthcare provider.

In step 420, when the oximetry paradoxus is determined to be equal to or larger than the threshold, the person can be confirmed to have pericardial tamponade by performing the confirmatory diagnostic testing for pericardial tamponade. By way of non-limiting example, the confirmatory diagnostic testing for pericardial tamponade includes pulsus paradoxus or echocardiography.

The disclosed thresholds may be derived by studying a derivation cohort and may be verified by studying a validation cohort.

Background: Although echocardiography is usually diagnostic of cardiac tamponade, it may not be readily available at the point-of-care. We sought to develop and validate a measurement of respirophasic variation in the amplitude of pulse oximetry plethysmographic waveforms as a diagnostic tool for cardiac tamponade.

Methods: Pulse oximetry plethysmographic waveforms were recorded and the ratio of maximum-to-minimum measured amplitude of these waveforms from one respiratory cycle was calculated by “blinded” observers; ratios from 3 consecutive respiratory cycles were then averaged to derive an “oximetry paradoxus” ratio. Cardiac tamponade was independently confirmed or excluded according to a “blinded” objective interpretation of echocardiography or right heart catheterization.

Results: Seventy-four subjects were enrolled (51% men; mean age 54±15 years); 19 of whom had cardiac tamponade. Oximetry paradoxus area under the curve (AUC) for diagnosis of cardiac tamponade was 0.90 (95% confidence interval, 0.84-0.97); its diagnostic performance was superior to sphygmomanometer-measured pulsus paradoxus (AUC difference=0.16, P=0.022). In a derivation cohort (N=37; tamponade, 9 cases), three diagnostic oximetry paradoxus thresholds were identified and validated in an independent validation cohort (N=37; tamponade, 10 cases): 1.2 (100% sensitivity, 44% specificity), 1.5 (80% sensitivity, 81% specificity), and 1.7 (80% sensitivity, 89% specificity). Furthermore, oximetry paradoxus was significantly reduced after draining pericardial fluid.

Conclusions: We defined and validated oximetry paradoxus as a simple and ubiquitous point-of-care test to diagnose cardiac tamponade using respirophasic changes in pulse plethysmography waveforms. This test can aid in identifying patients with cardiac tamponade, thus expediting confirmatory testing and life-saving treatment.

### Introduction

Pericardial tamponade is a life-threatening condition caused by a pathologic fluid accumulation in the pericardial space, leading to increased intra-pericardial pressure, compression of cardiac chambers, and eventual hemodynamic collapse. Arriving to a rapid diagnosis is essential for timely evacuation of the pericardial fluid to prevent or treat cardiovascular collapse. The clinical signs and symptoms of tamponade are nonspecific, but the diagnosis can be confirmed with echocardiography or right heart catheterization (RHC). Pulsus paradoxus, defined as excessive respiratory variation in the systolic blood pressure (≥10 mmHg), is considered pathognomonic [1, 2] but it is difficult to measure by inexperienced clinicians. Therefore, there is a need for a simple, rapid, reproducible, and ubiquitous bedside diagnostic tool to aid clinicians in identifying patients with evolving cardiac tamponade.

Plethysmographic waveform is a product of capillary volume which is dependent on stroke volume and arterial blood pressure. The amplitude of the plethysmography waveform varies with the respiratory cycle in conditions such as cardiac tamponade, establishing a graphic representation of pulsus paradoxus. It has been known that pulse oximetry plethysmographic waveforms demonstrate respirophasic variation in amplitude during severe asthma exacerbation and pericardial tamponade.[3, 4] A single retrospective case series of 12 patients with cardiac tamponade suggested that respiratory variation of pulse oximetry plethysmographic waveforms may have a value in diagnosing pericardial tamponade.5 However, the study included only a limited spectrum patients with severe cardiac tamponade and lacked a control group. Given the wide availability of pulse oximetry, measuring respirophasic changes in plethysmography waveforms provides a unique opportunity to identify patients with cardiac tamponade. In this investigation, we sought to identify and validate critical thresholds of respiratory variations in the amplitude of pulse plethysmography waveform to be used as a tool in diagnosing cardiac tamponade.

### Methods

Study Design:

A dual-center prospective study was conducted in sequential and separate Derivation and Validation Cohorts of patients with suspected or confirmed pericardial tamponade at the John H. Stroger, Jr. Hospital of Cook County (Chicago, Ill.) and Rush University Medical Center (Chicago, Ill.). Consenting patients underwent pulse oximetry plethysmography recording and a transthoracic echocardiogram or RHC to confirm or rule-out pericardial tamponade. Being a standard of care, no written consent was obtained for pulse oximetry evaluation. A HIPAA consent to collect personal health information was obtained. In critically ill or impaired patients who were unable to participate in the informed consent process, plethysmography data was collected and a proper consent to retain these data was obtained after clinical stabilization. The study was approved by the institutional review board in each participating institution.

Patients and Clinical Data

Patients with suspected cardiac tamponade referred for cardiology consultation or echocardiography examination were recruited. Identical exclusion criteria were applied in both cohorts, these were: 1) severe reactive airway disease with active wheezing;[2] 2) known pulmonary hypertension which can ameliorate tamponade physiology;[6] 3) acute pulmonary embolism, since it may mimic tamponade physiology;[7] 4) traumatic or post-surgical pericardial hematoma, since the hemodynamic characteristics and management of these conditions are different from medical tamponade.

Basic demographics, comorbidities, cancer history, and laboratory values were collected. The confirmed or presumed cause of pericardial effusion was tabulated. Subjects were examined by a senior cardiology fellow for jugular venous distention and pulsus paradoxus.

Pulse Oximetry Analysis

The pulse oximetry sensor was placed on the patient's index finger and pulse plethysmography waveforms were recorded using commercially available Passport 2-Datascope pulse oximetry device (Mindray, Shenzhen, China), shown in FIGS. 1, 2A-2D, and 5. Pulse plethysmography waveforms from ≥5 respiratory cycles were printed on gridded paper immediately before or after confirming the presence or absence of pericardial tamponade. Subsequently, the waveforms were analyzed off-line by observers blinded to the patient's clinical, echocardiography, or RHC data (GI, MJS). To ensure complete blinding, waveforms from each center were analyzed by an investigator affiliated with the partner institution.

The vertical amplitudes of plethysmography waveforms were measured (nadir to peak) and the ratio from of the maximum-to-minimum amplitude from one respiratory cycle was calculated. The ratio from three consecutive respiratory cycles were subsequently averaged and labeled as “oximetry paradoxus” (Ox-P). No decision or intervention was based on the assessment of pulse oximetry waveforms. For patients who underwent clinically-indicated pericardiocentesis or pericardial window, pulse plethysmography was repeated after pericardial fluid drainage.

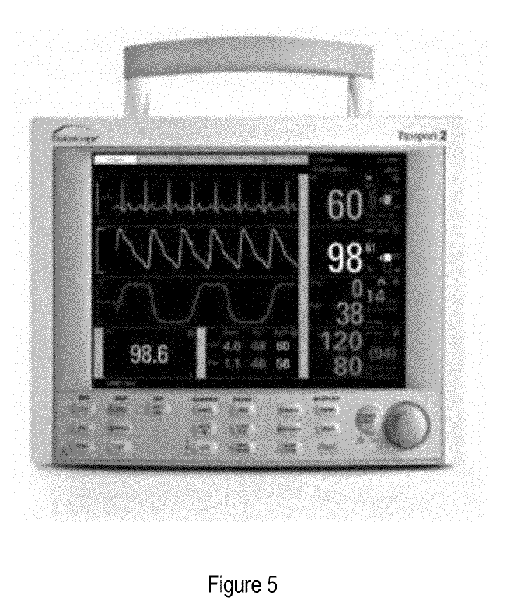

In order to demonstrate the reproducibility of Ox-P measurement, two observers (YG, CJB) independently measured Ox-P in 20 plethysmography recordings. Using two-way mixed model, the absolute interclass correlation agreement between observers was 0.987 (95% confidence interval [CI], 0.968-0.995), indicating excellent inter-rater agreementPulse oximetry sensor was placed on the patient's index finger or earlobe and the pulse oximetry waveform was recorded using commercially available Passport 2-Datascope pulse oximetry and cardiac monitor devise (Datascope Corp; Fairfield N.J.), FIG. 5. Waveform was printed and all measurements were performed on printed paper in a blinded fashion by an observer. Pulse oximetry waveform from ≥5 respiratory cycles was recorded before or after confirming the presence or absence of pericardial tamponade. Subsequently, the waveform was analyzed by an observer who was blinded to the patient's clinical or echocardiography data. The vertical amplitude of the pulse oximetry wave form was measured (from nadir to peak) and the ratio from of the maximum to the minimum amplitude within one respiratory cycle was calculated. The ratio from three consecutive respiratory cycles were subsequently averaged and labeled as “Oximetry Paradoxus”, which was then used in subsequent analysis. No decision or intervention was based on the assessment of pulse oximetry waveform. Among patients who underwent clinically-indicated pericardiocentesis or pericardial window, pulse oximetry waveform was repeated after pericardial fluid evacuation.

Clinical Data

The following clinical and laboratory data were collected: age, gender, basic cardiovascular risk factors and comorbidities (hypertension, diabetes mellitus, Hyperlipidemia, tobacco use, family history, chronic kidney disease stage), TSH, Free T4, blood urea nitrogen, and serum creatinine. Moreover, data of past or active cancer history and confirmed or presumed cause of pericardial effusion were tabulated. Each subject was examined by a senior cardiology fellow for jugular venous distention and pulsus paradoxus.

Pericardial Tamponade

The diagnosis of tamponade was confirmed based on clinical, echocardiographic, or RHC findings.

Clinical Criteria: defined as hypotension (systolic blood pressure<100 mmHg), tachycardia (heart rate≥100/min), and pulsus paradoxus (≥10 mmHg) or jugular venous distention, in association with echocardiographically proven pericardial effusion.

Echocardiographic Criteria: defined as echocardiographically proven pericardial effusion with one of the following criteria: a) right ventricular collapse>50% of the cardiac cycle; b) excessive (≥25%) respiratory variation in early mitral inflow E-wave velocity, as measured by pulsed-wave Doppler between the tips of the mitral valve leaflets, and inferior vena cava dilatation (≥2.1 mm) with absent respirophasic variation (<10%) by M-mode echocardiography; c) right atrial collapse>50% of the cardiac cycle and inferior vena cava dilatation (≥2.1 mm) with absent respirophasic variation (<10%), as measured by M-mode echocardiography. Echocardiography interpretations were performed in a blinded fashion by cardiologists (BM, CJB) specialized in echocardiography. To ensure complete blinding, echocardiography studies from each institution were interpreted by an investigator affiliated with the other participating institution.

RHC: defined as equalization (≤5 mmHg) of the right atrial pressure, right ventricular diastolic pressure, pulmonary arterial diastolic pressure, pulmonary capillary wedge pressure, and intra-pericardial pressure in the presence of echocardiographically confirmed pericardial effusion.

Data Fidelity and Blinding

To avoid any potential contamination between clinical data and the interpretation of pulse oximetry waveforms, echocardiography images, and right heart catheterization data, the interpretations of these studies were conducted by a physician affiliated with the institution other than the one where the patient presented or managed, such that data of patients presenting at John H. Stroger, Jr. Hospital of Cook County were interpreted by a physician affiliated with Rush University Medical Center and vice versa. Pulse oximetry waveforms were analyzed by senior cardiology fellows, one from each participating institution.

Derivation and Validation of Diagnostic Oximetry Paradoxus Thresholds

In the derivation cohort, a receiver operator characteristic (ROC) curve was used to determine the discriminatory capacity of Ox-P ratio, expressed as area under the curve (AUC) with CI. Based on the coordinates of the ROC curve, we determined a priori two desired Ox-P parameters: 1) ≥90% sensitivity threshold; 2) ≥80% specificity threshold. The diagnostic performance of each threshold was validated in the validation cohort using 2×2 contingency tables (test+/−versus disease+/−) to calculate sensitivity, specificity, positive predictive value, and negative predictive value.

Statistical Analyses

Sample size was estimated based on findings reported in in the case series by Stone et al.[5] We used pre-pericardiocentesis Ox-P values as “cases” (tamponade) and post-pericardiocentesis values as “controls”. The calculated mean Ox-P values for cases and controls were 1.91 and 1.22, respectively, and the pooled standard deviations (SD) was 0.51. Assuming cases to controls enrollment ratio of 1:2, we calculated a minimum of 9 cases are needed in each cohort to detect a statistically significant difference in the mean Ox-P between tamponade cases and controls using two-tailed independent samples t-test (power=0.90, α=0.05). To allow for error, we targeted a sample size of 36 subjects in each cohort (12 cases and 24 controls).

The chi-square test was used to compare dichotomous variables. The two-tailed independent-samples Student's t-test and the Wilcoxon test were used to compare normally-distributed and skewed continuous variables, respectively. The discriminatory capacities of various diagnostic parameters were compared using the AUC (C-statistic) and the chi-square test. The paired-samples two-tailed t-test was used to compare Ox-P values pre-versus-post pericardial fluid drainage. The SPSS 23 (IBM-Armonk, N.Y.) as used in data analysis, except for AUC comparisons which were performed using Stata 11 (StataCorp, LLC-College Station, Tex.).

### Results

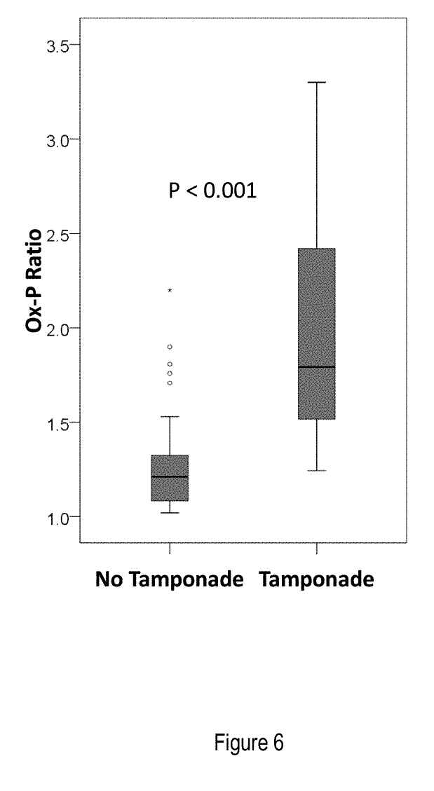

In the period between Jan. 2, 2013 and Jun. 9, 2014, 74 subjects with suspected or diagnosed pericardial tamponade were enrolled. The baseline characteristics of the study subjects were summarized in Table 1. All 74 subjects underwent transthoracic echocardiography assessment and 13 underwent RHC. One subject did not have echocardiography images available for blinded evaluation, but did undergo RHC confirming cardiac tamponade. A total of 19 (26%) subjects had evidence for pericardial tamponade, 7 by echocardiography and RHC, 9 by echocardiography alone, and 3 by RHC alone. One subject satisfied the clinical and echocardiographic criteria. Table 2 summarizes the clinical and echocardiographic findings in patients with and without pericardial tamponade. Notably, patients with pericardial tamponade had lower mean systolic blood pressure, higher mean heart rate, and higher prevalence of pulsus paradoxus. Patients with tamponade were more likely to be selected for RHC. The median Ox-P ratio was 1.28 (25th-75th percentile, 1.12-2.12; range, 1.02-3.30). As shown in Table 2 and FIG. 6, subjects with pericardial tamponade had statistically higher Ox-P ratio than those without evidence for tamponade (1.97±0.64 vs. 1.26±0.24, P<0.001).

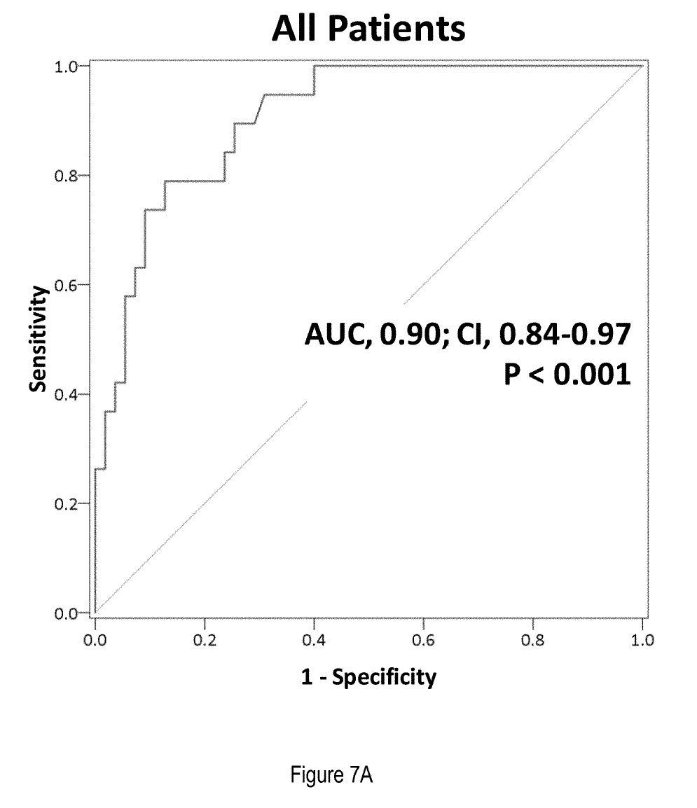

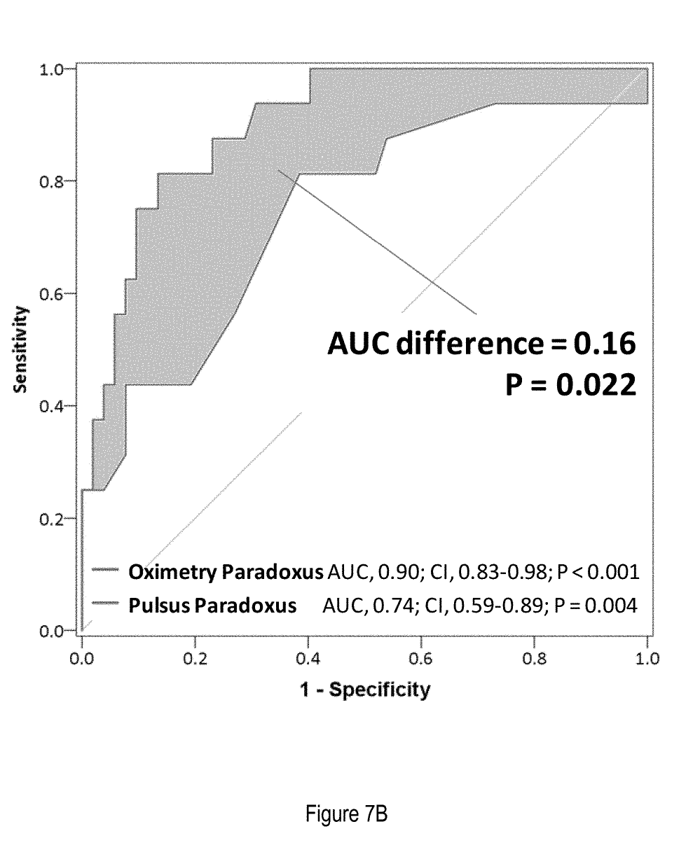

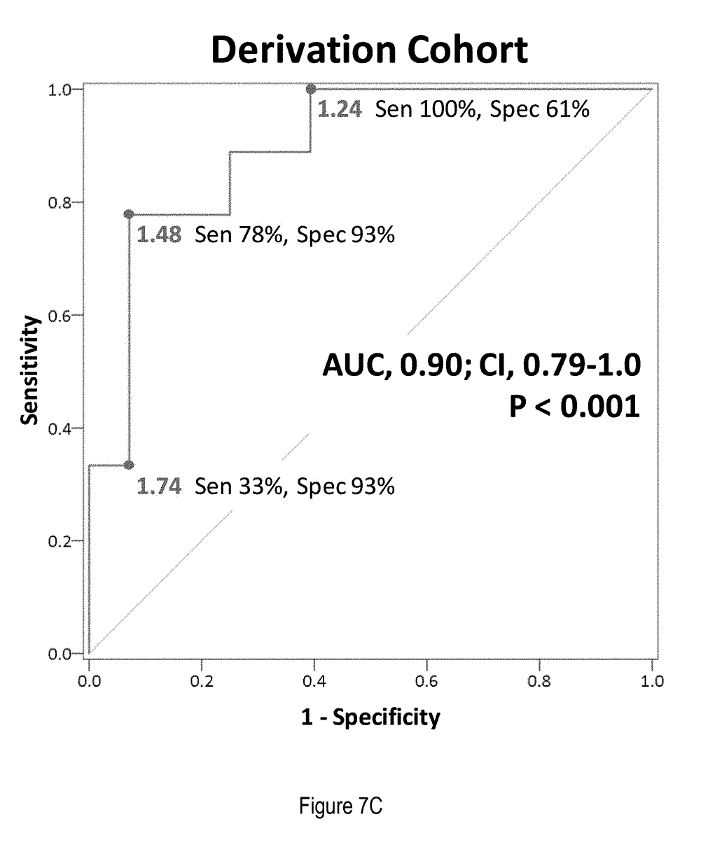

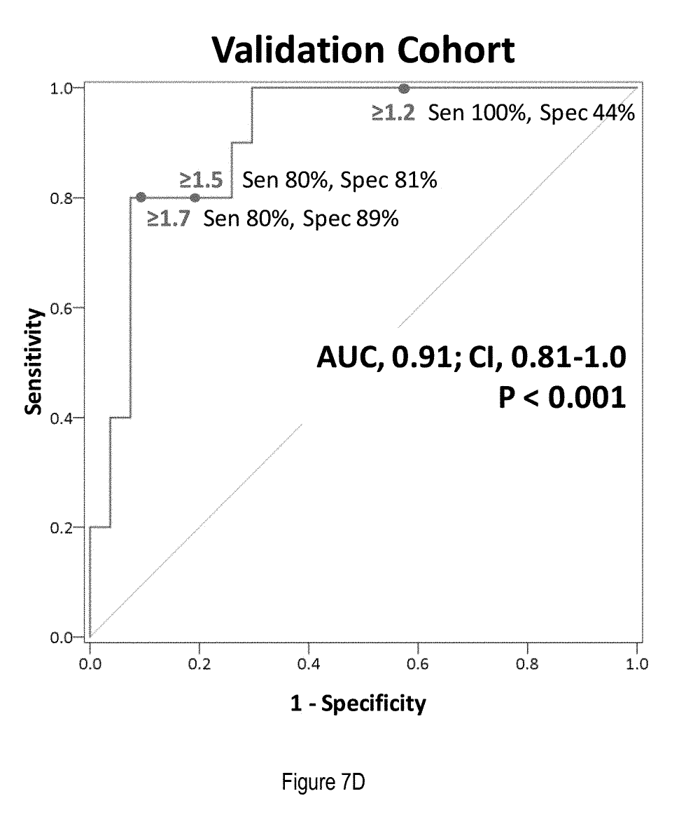

As shown in FIGS. 7A-7D, Ox-P ratio had an excellent discriminatory capacity for pericardial tamponade (AUC, 0.905; CI, 0.836-0.974; P<0.001). There was a moderate correlation between Ox-P ratios and pulsus paradoxus values (Pearson's r=0.59, P<0.001) and between Ox-P ratios and mitral valve inflow respirophasic variation (Pearson's r=0.58, P<0.001). Among 68 subjects who had pulsus paradoxus measurement, Ox-P ratio was associated with significantly greater discriminatory capacity than pulsus paradoxus (P=0.022), providing an incremental AUC of 0.16 (FIG. 7B). Moreover, there was no statistically significant difference in the discriminatory capacity of Ox-P (AUC, 0.90; CI, 0.83-0.97) and the mitral inflow E-wave respirophasic changes (AUC, 0.89; CI, 0.82-0.97).

TABLE 1
Baseline Characteristics of the Patient Population

| | | Derivation | Validation |
|---|---|---|---|
| | All Patients | Cohort | Cohort |
| Variables | N = 74 | N = 37 | N = 37 |
| Age, y | 54 ± 15 | 53 ± 14 | 54 ± 15 |
| Female | 36 (49) | 16 (43) | 20 (54) |
| Institution | | | |
| JHSHCC | 41 (55) | 22 (59) | 19 (51) |
| RUMC | 33 (45) | 15 (41) | 18 (49) |
| Medical History | | | |
| Hypertension | 39 (53) | 19 (51) | 17 (46) |
| Diabetes mellitus | 20 (27) | 10 (27) | 10 (27) |
| Heart failure | 25 (34) | 10 (27) | 15 (41) |
| Chronic kidney disease | 15 (20) | 7 (19) | 8 (22) |
| Dialysis therapy | 6 (8) | 2 (5) | 4 (11) |
| Hypothyroidism | 6 (8) | 3 (8) | 3 (8) |
| Connective tissue disease | 8 (11) | 5 (14) | 3 (8) |
| Pericarditis | 3 (4) | 2 (5) | 1 (3) |
| Lung cancer | 16 (22) | 8 (22) | 8 (22) |
| Breast cancer | 4 (5) | 1 (3) | 3 (8) |
| Other cancers | 15 (20) | 8 (22) | 7 (19) |
| Presenting Symptoms | | | |
| Dyspnea | 52 (70) | 26 (70) | 26 (70) |
| Fatigue | 22 (30) | 9 (24) | 13 (35) |
| Chest pain | 16 (22) | 10 (27) | 6 (16) |
| Dizziness | 13 (18) | 5 (14) | 8 (22) |
| Edema | 6 (8) | 1 (3) | 5 (14) |
| Syncope | 1 (1) | 0 (0) | 1 (3) |
| Suspected Etiology | | | |
| Malignancy | 29 (39) | 14 (38) | 15 (41) |
| Idiopathic | 10 (14) | 3 (8) | 7 (19) |
| Uremia | 10 (14) | 6 (16) | 4 (11) |
| CTD | 8 (10) | 5 (14) | 3 (8) |
| Heart failure | 6 (8) | 2 (5) | 4 (11) |
| Suspected viral | 4 (5) | 3 (8) | 1 (3) |
| Hypothyroidism | 2 (3) | 0 (0) | 2 (5) |
| Other | 5 (7) | 4 (11) | 1 (3) |
| SBP, mmHg | 129 ± 27 | 129 ± 29 | 128 ± 24 |
| DBP, mmHg | 76 ± 17 | 78 ± 18 | 74 ± 16 |
| Heart rate | 96 ± 22 | 95 ± 20 | 97 ± 23 |
| Respiratory rate | 22 ± 5 | 21 ± 4 | 22 ± 6 |
| O₂ saturation, % | 96 ± 3 | 96 ± 4 | 97 ± 2 |
| Pulsus paradoxus value, | 10.3 ± 7.4 | 9.1 ± 8.1 | 11.5 ± 6.5 |
| mmHg | | | |
| Pulsus paradoxus > 10 mmHg | 33 (45) | 11 (30) | 22 (59) |
| Jugular venous distention | 15 (20) | 7 (19) | 8 (22) |
| Creatinine, mg/dL | 1.8 ± 1.9 | 1.7 ± 1.7 | 2.0 ± 2.2 |
| Tamponade Diagnosis | | | |
| Any modality | 19 (26) | 9 (24) | 10 (27) |
| Echocardiography | 16 (22) | 8 (22) | 8 (22) |
| Right heart | 10 (14) | 5 (14) | 5 (14) |
| catheterization | | | |
| Clinical | 1 (1) | 0 (0) | 1 (3) |

CTD, connective tissue disease;
SBP, systolic blood pressure;
DBP, diastolic blood pressure;
Data presented as mean ± standard deviation or n (%)

TABLE 2
Baseline Clinical and Echocardiographic Characteristics of Patients with and
Without Pericardial Tamponade

| | | | No | |
|---|---|---|---|---|
| | All Patients | Tamponade | Tamponade | P |
| Variables | N = 74 | N = 19 | N = 55 | value |
| Age, y | 54 ± 15 | 52 ± 11 | 54 ± 16 | 0.666 |
| Female | 36 (49) | 10 (53) | 26 (47) | 0.687 |
| Institution | | | | |
| JHSHCC | 41 (55) | 14 (74) | 27 (49) | 0.064 |
| RUMC | 33 (45) | 5 (26) | 28 (51) | |
| Symptoms | 67 (91) | 49 (89) | 18 (95) | 0.468 |
| Malignant effusion | 29 (39) | 12 (63) | 17 (31) | 0.013 |
| Uremic effusion | 10 (14) | 0 (0) | 10 (18) | 0.046 |
| SBP, mmHg | 129 ± 27 | 117 ± 17 | 133 ± 28 | 0.006 |
| Heart rate | 96 ± 22 | 107 ± 20 | 92 ± 21 | 0.009 |
| Respiratory rate | 22 ± 5 | 23 ± 4 | 21 ± 5 | 0.283 |
| O₂ saturation, % | 96 ± 3 | 96 ± 3 | 97 ± 3 | 0.488 |
| Pulsus paradoxus value, | 10.3 ± 7.4 | 15.8 ± 11.2 | 8.6 ± 4.7 | 0.023 |
| mmHg | | | | |
| Pulsus paradoxus > 10 mmHg | 33 (49) | 13 (81) | 20 (38) | 0.003 |
| Jugular venous distention | 15 (21) | 5 (26) | 10 (19) | 0.469 |
| Electrical Alternans | 8 (11) | 4 (21) | 4 (8) | 0.115 |
| Creatinine, mg/dL | 1.8 ± 1.9 | 1.1 ± 1.0 | 2.1 ± 2.1 | 0.011 |
| MV E velocity variation, % | 24 ± 11 | 36 ± 9 | 20 ± 9 | <0.001 |
| MV E velocity variation > 25% | 29 (40) | 17 (94) | 12 (22) | <0.001 |
| TV E velocity variation, % | 34 ± 12 | 38 ± 11 | 33 ± 12 | 0.134 |
| TV E velocity variation > 40% | 15 (28) | 6 (43) | 9 (23) | 0.159 |
| IVC diameter ≥ 2.1 cm | 28 (38) | 13 (72) | 15 (27) | 0.001 |
| Blunted IVC collapse (<10%) | 24 (33) | 12 (67) | 12 (22) | <0.001 |
| RHC performed | 13 (18) | 11 (58) | 2 (4) | <0.001 |
| Oximetry paradoxus (Ox-P) ratio | 1.4 ± 0.5 | 1.97 ± 0.64 | 1.26 ± 0.24 | <0.001 |
| Mean ± SD | 1.28 (1.12-1.56) | 1.79 (1.49-1.79) | 1.21 (1.08-1.33) | |
| Median (25th-75th percentile) | | 1.24-3.3 | 1.02-2.2 | |
| Range (min-max) | 1.02-3.30 | | | |

CTD, connective tissue disease;
SBP, systolic blood pressure;
DBP, diastolic blood pressure;
MV, mitral valve;
E, early diastolic mitral or tricuspid inflow velocity by pulsed wave Doppler;
TV, tricuspid valve;
IVC, inferior vena cava;
JHSHCC, John H. Stroger, Jr. Hospital of Cook County;
RUMC, Rush University Medical Center;
RHC, right heart catheterization
Data presented as mean ± standard deviation or n (%)

Derivation and Validation of Ox-P Diagnostic Thresholds

The derivation cohort consisted of 37 subjects, among whom 9 met study criteria for pericardial tamponade. As illustrated in FIG. 7C, Ox-P ratio was associated with an AUC of 0.90 (CI, 0.79-1.0; P<0.001) for cardiac tamponade. Examination of the ROC curve coordinates yielded two Ox-P values meeting the a priori defined diagnostic performance; these were 1.24 (sensitivity≥90%) and 1.48 (specificity≥80%). We also identified a higher threshold of 1.74, as a potential higher specificity cutoff. For simplicity, these thresholds were rounded to the nearest 1 decimal place value.

The validation cohort consisted of 37 subjects, among whom 10 met the study criteria for cardiac tamponade. As illustrated in FIG. 7D, Ox-P ratio had an AUC of 0.90 (CI, 0.81-1.0; P<0.001). Two-by-two contingency tables were constructed to validate each threshold identified in the derivation cohort. As shown in Table 3, Ox-P≥1.2 was confirmed as a highly sensitive threshold, while ≥1.5 and ≥1.7 were confirmed as highly specific.

TABLE 3
Validation of Ox-P Ration
Diagnostic Thresholds

| | | |
|---|---|---|
| Tamponade | Tamponade | |
| Absent | Present | Diagnostic |
| N = 27 (73) | N = 10 (27) | Performance |

Threshold 1.2

| | | | | |
|---|---|---|---|---|
| Ox-P ratio < 1.2 | 12 (44) | 0 (0) | Sensitivity | 100% |
| N = 12 (32) | | | Specificity | 44% |
| | | | PPV | 40% |
| Ox-P ratio ≥ 1.2 | 15 (56) | 10 (100) | NPV | 100% |
| N = 25 (68) | | | Accuracy | 59% |
| | | | P value | 0.010 |

Threshold 1.5

| | | | | |
|---|---|---|---|---|
| Ox-P ratio < 1.5 | 22 (81) | 2 (20) | Sensitivity | 80% |
| N = 24 (65) | | | Specificity | 81% |
| | | | PPV | 62% |
| Ox-P ratio ≥ 1.5 | 5 (19) | 8 (80) | NPV | 81% |
| N = 13 (35) | | | Accuracy | 81% |
| | | | P value | 0.001 |

Threshold 1.7

| | | | | |
|---|---|---|---|---|
| Ox-P ratio < 1.7 | 24 (89) | 2 (20) | Sensitivity | 80% |
| N = 26 (70) | | | Specificity | 89% |
| | | | PPV | 73% |
| Ox-P ratio ≥ 1.7 | 3 (11) | 8 (80) | NPV | 92% |
| N = 11 (30) | | | Accuracy | 86% |
| | | | P value | <0.001 |

Two-by-two contingency table for E/e′ ≥ 8.0 versus < 8.0 test status and LAA thrombus status
Data is presented as frequencies (%)
Sensitivity = 100%,
Specificity = 41%,
Positive Predictive Value = 10%,
Negative Predictive Value = 100%,
Positive Likelihood Ratio = 1.69
Negative Likelihood Ratio = 0

Impact of Pericardial Fluid Evacuation on Ox-P Ratio

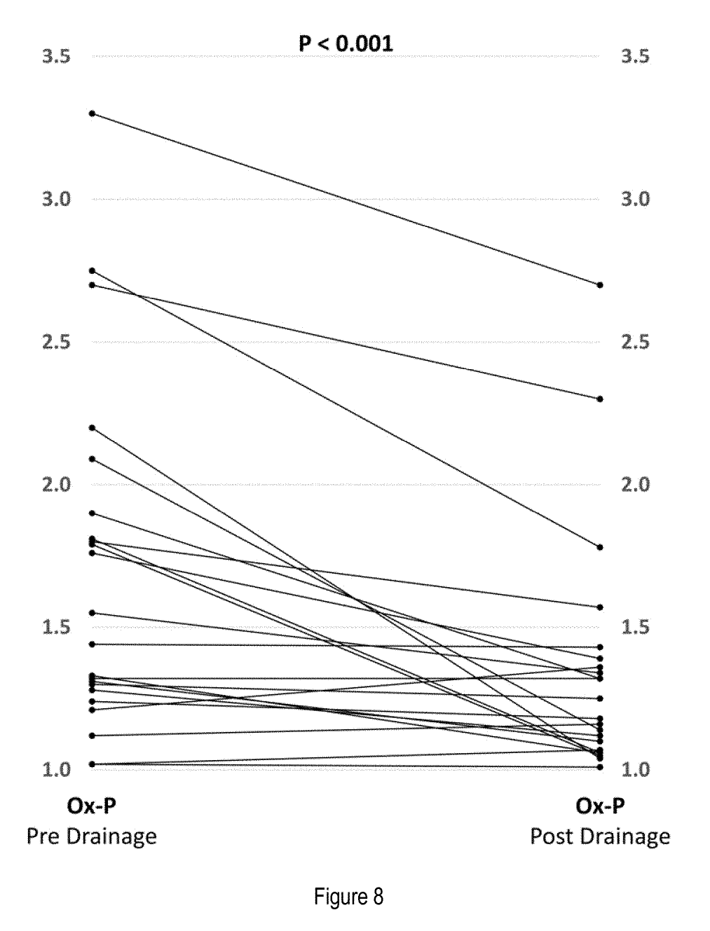

Among all study subjects, 35 with or without tamponade had pericardial fluid drainage either percutaneously (n=18) or surgically (n=17). The median fluid volume drained was 680 mL (25th-75th percentile, 500-900 mL). Among 22 subjects who had Ox-P measured after initial fluid drainage, the mean Ox-P decreased from a mean of 1.69±0.61 pre-drainage to 1.35±0.42 post-drainage (P<0.001), a mean decline of 0.34±0.38 (FIG. 8). Among the 11 patients with tamponade, Ox-P decreased from 1.93±0.70 pre-drainage to 1.52±0.54 immediately post-drainage (P=0.004), a mean decline of 0.41±0.36.

### Discussion

We prospectively derived and validated oximetry paradoxus as an objective and reproducible diagnostic test for cardiac tamponade. We identified an Ox-P of ≥1.2 to be highly sensitive for cardiac tamponade, thus a value <1.2 can effectively “rule-out” this life-threatening condition, particularly when the pretest likelihood is low. On the other hand, an Ox-P≥1.5 should raise suspicion of cardiac tamponade and trigger confirmatory testing. Our investigation proposes “oximetry paradoxus” as a simple and ubiquitous diagnostic tool for cardiac tamponade.

Clinical diagnosis of cardiac tamponade is challenging. The clinical presentation is often vague with non-specific symptoms, such as dyspnea and chest discomfort.⁸ Physical exam findings are non-specific either and may include hypotension, tachycardia, tachypnea, jugular venous distension, and peripheral edema.⁹ Pulsus paradoxus, defined as the exaggerated decline in systolic blood pressure during inspiration ≥10 mmHg, is a more specific sign of cardiac tamponade.[2, 9, 10] The “paradox” refers to the heart sounds being heard over the precordium when the radial pulse is not felt. In the normal state, during inspiration, more blood returns to the right heart and pools in the expanded pulmonary vasculature. Blood pooling in the pulmonary circulation decreases the blood return to the left heart, leading to a slight decrease in the systemic stroke volume and systolic blood pressure (<10 mmHg) during inspiration. In cardiac tamponade, due to external constrain from pericardial pressure, the inspiratory increase in venous return cannot be accommodated by expansion of the right heart, but rather by shifting the interventricular septum to towards the left ventricle, decreasing left ventricular filling which further diminishes the stroke volume and systolic blood pressure. During the subsequent expiration, decreased systemic venous return to the right heart along with the return of the blood

pooled in the pulmonary circulation to left heart lead to augmentation in systemic stroke volume and systolic blood pressure, thus exaggerating the respirophasic variation in systolic blood pressure to ≥10 mmHg.² Pulsus paradoxus can be measured while deflating the sphygmomanometer cuff by calculating the difference in systolic blood pressure of when Korotkoff sounds are initially heard in expiration only to when those sounds are heard consistently in inspiration and expiration upon further cuff deflation. Measuring pulsus paradoxus requires significant clinical skills. On the other hand, pulse oximetry is a standard element of the evaluation of acutely ill patients; thus, measuring Ox-P can be easily performed as part of a routine assessment of these patients. Our study demonstrated that Ox-P measurement is not only simple and reproducible, but also more accurate than pulsus paradoxus and as accurate as Doppler echocardiography. These findings establish the foundation for Ox-P as a powerful bedside tool in the assessment of patients with suspected cardiac tamponade.

It is established that acute bronchospasm can cause respirophasic changes in systemic stroke volume manifesting as pulsus paradoxus and increased Ox-P ratio.² Hence, Ox-P values should be always interpreted in light of the clinical context. Moreover, pericardial fluid seen on point-of-care two-dimensional echocardiography or enlarged cardiac silhouette on chest x-ray can point out to cardiac tamponade as an etiology for high Ox-P value, while lung exam and spirometry would clearly direct attention towards acute bronchospasm.

It is important to note that the Ox-P thresholds we derived from a specific commercially available device. However, since our Ox-P measurement is based on wave amplitude ratio rather than

absolute values, it is expected for devices from other manufacturer to produce similar results, but this assumption should be validated.

In the future, the measurement of Ox-P can be automated using waveform analysis software embedded in the pulse oximetry equipment. Automatic warnings can be triggered when the Ox-P ratio exceeds a set threshold in order to alert healthcare providers of the possibility of cardiac tamponade.

### Limitations

The small sample size is an obvious limitation. This study excluded patients with acute bronchospasm that may cause respirophasic changes in pulse volume and hence high Ox-P ratio. The clinical context, lung exam, chest X-ray, and two-dimensional echocardiography can help differentiate between these distinct clinical entities.

### Conclusions

We defined and validated oximetry paradoxus as a simple and ubiquitous point-of-care test to screen for cardiac tamponade using respirophasic changes in the amplitude of pulse plethysmography waveforms. Identifying patients with likely cardiac tamponade using this method can trigger confirmatory testing and expedite the implementation of life-saving treatment.

### REFERENCES

1. Roy C L, Minor M A, Brookhart M A, Choudhry N K. Does this patient with a pericardial effusion have cardiac tamponade? Jama. 2007; 297(16):1810-1818.
2. Hamzaoui O, Monnet X, Teboul J L. Pulsus paradoxus. Eur Respir J. 2013; 42(6):1696-1705.
3. Frey B, Butt W. Pulse oximetry for assessment of pulsus paradoxus: a clinical study in children.
Intensive Care Med. 1998; 24(3):242-246.
4. Amoozgar H, Ghodsi H, Borzoee M, Amirghofran A A, Ajami G, Serati Z. Detection of pulsus paradoxus by pulse oximetry in pediatric patients after cardiac surgery. Pediatric cardiology. 2009; 30(1):41-45.
5. Stone M K, Bauch T D, Rubal B J. Respiratory changes in the pulse-oximetry waveform associated with pericardial tamponade. Clin Cardiol. 2006; 29(9):411-414.
6. Frey M J, Berko B, Palevsky H, Hirshfeld J W, Jr., Herrmann H C. Recognition of cardiac tamponade in the presence of severe pulmonary hypertension. Ann Intern Med. 1989; 111(7):615-617.
7. McDonald I G, Hirsh J, Jelinek V M, Hale G S. Acute major pulmonary embolism as a cause of exaggerated respiratory blood pressure variation and pulsus paradoxus. Br Heart J. 1972; 34(11):1137-1141.
8. Spodick D H. Acute cardiac tamponade. N Engl J Med. 2003; 349(7):684-690.
9. Guberman B A, Fowler N O, Engel P J, Gueron M, Allen J M. Cardiac tamponade in medical patients. Circulation. 1981; 64(3):633-640.
10. McGregor M. Current concepts: pulsus paradoxus. N Engl J Med. 1979; 301(9):480-482.

While the particular invention has been described with reference to illustrative embodiments, this description is not meant to be limiting. Various modifications of the illustrative embodiments and additional embodiments of the invention will be apparent to one of ordinary skill in the art from this description. Those skilled in the art will readily recognize that these and various other modifications can be made to the exemplary embodiments, illustrated and described herein, without departing from the spirit and scope of the present invention. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

1. A method for diagnosing pericardial tamponade, comprising:
analyzing, by a device comprising a memory and a processor in communication with the memory, a pulse oximetry waveform of a person to obtain an oximetry paradoxus, wherein the analyzing the pulse oximetry waveform to obtain the oximetry paradoxus comprises:
identifying, by the device, a maximum amplitude and a minimum amplitude of the pulse oximetry waveform within a respiratory cycle, and
calculating, by the device, a ratio of the maximum amplitude and the minimum amplitude within the respiratory cycle to obtain the oximetry paradoxus;
determining, by the device, whether the oximetry paradoxus is equal to or larger than a threshold; and
when the oximetry paradoxus is determined to be equal to or larger than the threshold, identifying, by the device, the person as having or suspected as having pericardial tamponade.

2. The method of claim 1, wherein
the pulse oximetry waveform comprises one or more respiratory cycles.

3. The method of claim 1, wherein the pulse oximetry waveform within the respiratory cycle comprises at least two cardiac cycles.

4. The method of claim 1, wherein the analyzing the pulse oximetry waveform to obtain the oximetry paradoxus comprises:
identifying, by the device, a maximum amplitude and a minimum amplitude of the pulse oximetry waveform within each respiratory cycle of at least two respiratory cycles in the pulse oximetry waveform;
calculating, by the device, a ratio of the maximum amplitude and the minimum amplitude within the each respiratory cycle; and
calculating, by the device, an average of the ratio within the each respiratory cycle to obtain the oximetry paradoxus.

5. The method of claim 4, wherein the pulse oximetry waveform within the each respiratory cycle comprises at least two cardiac cycles.

6. The method of claim 1, wherein the analyzing the pulse oximetry waveform to obtain the oximetry paradoxus comprises:
selecting, by the device, three respiratory cycles from at least three respiratory cycles in the pulse oximetry waveform;
identifying, by the device, a maximum amplitude and a minimum amplitude of the pulse oximetry waveform within each respiratory cycle of the three respiratory cycles;
calculating, by the device, a ratio of the maximum amplitude and the minimum amplitude within the each respiratory cycle; and
calculating, by the device, an average of the ratio within the each respiratory cycle to obtain the oximetry paradoxus.

7. The method of claim 6, wherein the three respiratory cycles are consecutive.

8. The method of claim 1, wherein, when the oximetry paradoxus is determined to be equal to or larger than the threshold, the identifying the person as having or suspected as having pericardial tamponade comprises:
identifying, by the device, the person as having or suspected as having pericardial tamponade with a sensitivity and a specificity.

9. The method of claim 8, wherein:
the threshold is about 1.24, the sensitivity is about 100%, and the specificity is about 61%.

10. The method of claim 8, wherein:
the threshold is about 1.48, the sensitivity is about 78%, and the specificity is about 93%.

11. The method of claim 8, wherein:
the threshold is about 1.74, the sensitivity is about 33%, and the specificity is about 93%.

12. The method of claim 1, further comprising:
outputting, by the device, a warning signal to alert a healthcare provider that the person is identified as having or suspected as having pericardial tamponade when the oximetry paradoxus is determined to be equal to or larger than the threshold.

13. The method of claim 12, further comprising:
providing, by the device, an audio signal or a video signal as the warning signal.

14. The method of claim 1, further comprising:
performing a confirmatory diagnostic test for pericardial tamponade when the oximetry paradoxus is determined to be equal to or larger than the threshold.

15. The method of claim 14, further comprising:
performing pulsus paradoxus or echocardiography as the confirmatory diagnostic test.

16. A device for diagnosing pericardial tamponade, comprising:
a memory storing instructions; and
a processor in communication with the memory, wherein, when the processor executes the instructions, the processor is configured to cause the device to:
identify a maximum amplitude and a minimum amplitude of a pulse oximetry waveform of a person within a respiratory cycle, and
calculate a ratio of the maximum amplitude and the minimum amplitude within the respiratory cycle to obtain an oximetry paradoxus;
determine whether the oximetry paradoxus is equal to or larger than a threshold, and
when the oximetry paradoxus is determined to be equal to or larger than the threshold, identify the person as having or suspected as having pericardial tamponade.

17. The device of claim 16, wherein the
the pulse oximetry waveform comprises one or more respiratory cycles.

18. The device of claim 16, wherein, when the processor is configured to cause the device to calculate the ratio of the maximum amplitude and the minimum amplitude to obtain the oximetry paradoxus, the processor is configured to cause the device to:
identify the maximum amplitude and the minimum amplitude of the pulse oximetry waveform within each respiratory cycle of at least two respiratory cycles in the pulse oximetry waveform;
calculate the ratio of the maximum amplitude and the minimum amplitude within the each respiratory cycle; and
calculate an average of the ratio within the each respiratory cycle to obtain the oximetry paradoxus.

19. A non-transitory computer readable storage medium comprising instructions for diagnosing pericardial tamponade, wherein, the instructions, when executed by a processor in a device, direct the device to:
identify a maximum amplitude and a minimum amplitude of a pulse oximetry waveform of a person within a respiratory cycle;
calculate a ratio of the maximum amplitude and the minimum amplitude within the respiratory cycle to obtain an oximetry paradoxus;
determine whether the oximetry paradoxus is equal to or larger than a threshold; and
when the oximetry paradoxus is determined to be equal to or larger than the threshold, identify the person having pericardial tamponade.

20. The non-transitory computer readable storage medium of claim 19, wherein, when the instructions are configured to cause the device to calculate the ratio of the maximum amplitude and the minimum amplitude to obtain the oximetry paradoxus, the instructions are configured to cause the device to:
identify the maximum amplitude and the minimum amplitude of the pulse oximetry waveform within each respiratory cycle of at least two respiratory cycles in the pulse oximetry waveform;
calculate the ratio of the maximum amplitude and the minimum amplitude within the each respiratory cycle; and
calculate an average of the ratio within the each respiratory cycle to obtain the oximetry paradoxus.
